(19) United States
(12) Patent Application Publication
(10) Pub. No.: US 2012/0064525 A1
(43) Pub. Date: Mar. 15, 2012

(54) METHOD FOR COLLECTION OF NUCLEIC ACID DERIVED FROM MAMMALIAN CELL, METHOD FOR ANALYSIS OF NUCLEIC ACID, AND KIT FOR COLLECTION OF FECES

(75) Inventors: Masanori Asakura, Tokyo (JP); Tomonori Nagaoka, Tokyo (JP); Yasuo Tanigami, Tokyo (JP)

(73) Assignee: OLYMPUS CORPORATION, TOKYO (JP)

(21) Appl. No.: 13/300,123

(22) Filed: Nov. 18, 2011

Related U.S. Application Data
(63) Continuation of application No. PCT/JP2010/001750, filed on Mar. 11, 2010.

(30) Foreign Application Priority Data
May 20, 2009 (JP) 2009-122439

(51) Int. Cl. C12Q 1/68; C07H 21/00; C12M 1/00; C07H 1/08

(52) U.S. Cl. 435/6.11; 536/25.4; 536/23.5; 435/6.1; 435/6.14; 435/6.18; 435/283.1; 435/6.12

(57) ABSTRACT

A method of recovering a nucleic acid derived from a mammalian cell taken from feces is provided. The nucleic acid is recovered easily even from feces collected in routine health checkups or the like. The nucleic acid of mammalian cell can be recovered more selectively than that from an enterobacterial cell. The method of recovering of the present invention includes the steps of, (A) preparing a fecal sample by placing the feces in a treatment solution with a high salt concentration, immersing the feces in the treatment solution for a predetermined period; (B) recovering a solid component from the fecal sample after the step (A); and (C) recovering a nucleic acid from the solid component recovered in the step (B).

This application is a continuation application based on a PCT Patent Application No. PCT/JP2010/001750, filed Mar. 11, 2010, whose priority is claimed on Japanese Patent Application No. 2009-122439, filed May 20, 2009. The content of both the PCT Application and the Japanese Application are incorporated herein by reference.

### BACKGROUND OF THE INVENTION

1. Field of the Invention

The present invention relates to a method of recovering a nucleic acid derived from a mammalian cell taken from feces more selectively than a nucleic acid derived from an enterobacterial cell, a method of analyzing a nucleic acid using the nucleic acid recovered by the recovering method above, and a kit for feces sampling suitable for the recovering method above.

2. Description of Related Art

In Japan as well as in Europe, the number of patients with colorectal cancer has been increasing rapidly year by year. As a result, cancer mortality due to the colon cancer has been ranked in the top list. It is believed that the reason for this is due to a change in the eating habits of Japanese to the western style diet where a major component of the diet is meat. Specifically, about 60,000 people suffer from colon cancer each year. Regarding the number of deaths in terms of cancer in different organs, colon cancer is the third highest after stomach cancer and lung cancer. The number of deaths from colon cancer is predicted to increase further in the future. On another front, colon cancer, unlike other cancers, can be cured by nearly 100% by treatment at the early stage of the occurrence of the disease. Therefore, it is extremely important to screen for colon cancer in an initial cancer screening. As a result, research and development of a testing method for early detection of colorectal cancer have been actively conducted.

As testing methods for early detection of colorectal cancer, an enema, a colonoscopy, or the like has been performed. During an enema, barium is injected into the colon, and is deposited on the surface of the lining of the colon. Then, images of recesses and protrusions on the surface are taken by X-ray. In this way, the colon surface is visually examined. During a colonoscopy, the inside of the colon is directly observed with an endoscopic instrument. Particularly, during the colonoscopy, colon cancer can be detected highly sensitivity and specifically. Furthermore, polyps and early cancers can be resected in the colonoscopy.

Recently, as a primary screening method for colorectal cancer, the fecal occult blood test has been widely performed. In the fecal occult blood test, the cost for the test is inexpensive, and the test is not invasive. The fecal occult blood test is a test examining the presence of hemoglobin from red blood cells contained in the feces. By the test, the presence of colon cancer can be predicted indirectly. In the fecal occult blood test, sampling and storing of the feces can be performed at room temperature. Also, a special storage condition of the samples is not required. Also, the test sampling can be performed at a normal household. Also, the operations in the sampling and storing are straightforward. Due to the reasons described above, the fecal occult blood test has been performed widely.

A new testing method, which is suitable for a regular checkups, noninvasive, simple to operate, and capable of a highly reliable diagnosis, has been drawing attention recently. In the new testing method, the existence or non-existence of cancer cells or genes derived from cancer cells in the feces is examined. Since the existence or non-existence of cancer cells or genes derived from cancer cells is examined directly, a highly reliable diagnosis is possible in the method compared to the fecal occult blood test. In the fecal occult blood test, bleeding from a gastrointestinal tract, which is an indirect consequence of having the colon cancer, is examined.

In order to accurately detect cancer cells or the like in feces samples, it is critical to recover efficiently the nucleic acid derived from the cancer cells in the feces sample. Particularly, the amount of the nucleic acid derived from the cancer cells is extremely small. Furthermore, the nucleic acid can be easily degraded because there are a lot of digested residues and bacteria in feces. Thus, it is important to prevent the degradation of the nucleic acid, particularly one derived from a mammalian cell, such as a human cell. Furthermore, it is important to prepare and store the feces samples in a stable state by time for the operation of the testing. As a method of preparing the feces samples in a stable state, for example, there is a method, in which cancer cells exfoliated from colorectal and other gastrointestinal tract are isolated from the sampled feces. By separating cancer cells from the feces, the effects of degrading enzymes, such as protease, DNase, RNase, or the like derived from bacteria or the like, can be suppressed. As a method of isolating cancer cells from feces, a method disclosed below. The method (1) comprises the steps of a) cooling the feces lower than its gel freezing point, and b) collecting cells in a condition where the feces is kept at a temperature lower than the gel freezing point to keep the feces in a substantially intact form (see Published Japanese Translation No. H11-511982 of the PCT International Publication). Also, a method (2), in which the colonic exfoliated cells are isolated after dispersing them at an ambient temperature in a transport medium containing a protease inhibitor, a mucolytic agent, and a bactericidal agent, is disclosed (see Published Japanese Translation No. 2004-519202 of the PCT International Publication).

In the other disclosed method (3), the fecal sample is treated in a special buffer containing about 10 to 200 mM of a chelator, 1 to 20 mM of a salt, and at least about 500 mM of a buffer, the pH of the special buffer being set to about 8.0 to 9.0 (see Japanese Patent (Granted) Publication No. 3633932). In the treatment, a non-particulate fraction is formed from the fecal sample and the special buffer. A nucleic acid derived from a mammalian cell is recovered from the non-particulate fraction. In the special buffer, the degradation of the nucleic acid is minimized by its pH and chelating capability. Furthermore, not bacterial cells, but eukaryotic cells are dissolved. As a result, the nucleic acid derived from eukaryotic cells is selectively extracted in the non-particulate fraction.

On another front, as a method of storing a biological sample stably before nucleic acid extraction, the method (4), in which gene induction in vitro is prevented by contacting a collected whole blood sample to a stabilizing additive immediately, is disclosed. In the method (4), the transcriptional profile in vitro is kept un-interfered. As a stabilizing additive, a detergent, chaotropic salt, a ribonuclease suppressor, a chelating agent, or a mixture thereof, an organic solvent, or an organic reducing agent can be used (see Published Japanese Translation No. 2004-534731 of the PCT International Publication). In the other disclosed method (5), RNAs in a cell and a sample tissue are protected from nucleases by immersing the samples containing RNAs in an RNA storing solution. The RNA storing solution contains a salt, a chelating agent that chelates a divalent cation, or a buffer whose pH is 4 to 8 (see Published Japanese Translation No. 2002-521071). In addition, the method (6), in which nuclease activity in the sample is suppressed, is also disclosed (see Japanese Unexamined Patent Application, First Publication No. 2007-151470). The method (6) comprises the first step, the second step, and the third step. In the first step, the sample is added to a sample storing solution including a salt of a monovalent cation as an active component. In the second step, all or part of the mixture obtained in the first step is added to a nucleic acid amplification system. In the third step, a nucleic acid amplification reaction is performed.

### SUMMARY OF THE INVENTION

As aspects of the present invention, methods of recovering a nucleic acid, methods of analyzing a nucleic acid, kits for feces sampling, and a fecal sample processing apparatus indicated below are provided.

(1) A method of recovering a nucleic acid derived from a mammalian cell taken from feces comprising the steps of: (A) immersing feces, in which feces is immersed in a treatment solution with a high salt concentration for a predetermined period of time to prepare a fecal sample; (B) recovering a solid component, in which a solid component is recovered from the fecal sample after the step (A); and (C) recovering a nucleic acid from the solid component recovered in the step (B).

(2) The method of recovering a nucleic acid derived from a mammalian cell taken from feces described in the above-mentioned (1), wherein, the treatment solution with a high salt concentration is a solution containing one or more salts selected from the group consisting of sodium chloride and ammonium sulfate.

(3) The method of recovering a nucleic acid derived from a mammalian cell taken from feces described in the above-mentioned (1), wherein, the treatment solution with a high salt concentration contains sodium chloride, the concentration of which is 13% (wt/wt) or higher and the saturating concentration of sodium chloride or lower.

(4) The method of recovering a nucleic acid derived from a mammalian cell taken from feces described in the above-mentioned (1), wherein, the treatment solution with a high salt concentration contains sodium chloride, the concentration of which is 20% (wt/wt) or higher and the saturating concentration of sodium chloride or lower.

(5) The method of recovering a nucleic acid derived from a mammalian cell taken from feces described in the above-mentioned (1), wherein, the treatment solution with a high salt concentration contains ammonium sulfate, the concentration of which is 30% (wt/wt) or higher and the saturating concentration of ammonium sulfate or lower.

(6) The method of recovering a nucleic acid derived from a mammalian cell taken from feces described in the above-mentioned (1), wherein, a salt concentration of the treatment solution with a high salt concentration is 80% of the saturating concentration of the salt or higher and the saturating concentration of the salt concentration or lower.

(7) The method of recovering a nucleic acid derived from a mammalian cell taken from feces described in the above-mentioned (1), wherein, a salt concentration of the treatment solution is the saturating concentration of the salt.

(8) The method of recovering a nucleic acid derived from a mammalian cell taken from feces described in the above-mentioned (1), wherein, the treatment solution with a high salt concentration contains two or more kinds of salts.

(9) The method of recovering a nucleic acid derived from a mammalian cell taken from feces described in the above-mentioned (1), wherein, the treatment solution with a high salt concentration contains a chelating agent that complexes with a divalent cation.

(10) The method of recovering a nucleic acid derived from a mammalian cell taken from feces described in the above-mentioned (1), wherein, the pH value of the treatment solution with a high salt concentration ranges from 4 to 8.

(11) The method of recovering a nucleic acid derived from a mammalian cell taken from feces described in the above-mentioned (1), wherein, the treatment solution with a high salt concentration has a buffer action.

(12) The method of recovering a nucleic acid derived from a mammalian cell taken from feces described in the above-mentioned (1), further comprising the step of: (D) washing the solid component recovered in the step (B) to reduce the salt concentration of the solid component, after the step (B) and before the step (C).

(13) The method of recovering a nucleic acid derived from a mammalian cell taken from feces described in the above-mentioned (12), wherein, the salt concentration of the solid component after the step (D) is less than 100 mM.

(14) The method of recovering a nucleic acid derived from a mammalian cell taken from feces described in the above-mentioned (12), wherein, the salt concentration of the solid component after the step (D) is less than 30 mM.

(15) The method of recovering a nucleic acid derived from a mammalian cell taken from feces described in the above-mentioned (12), wherein, the step (D) comprises the steps of: (d) dispersing the solid component in a washing solution; and (e) recovering a washed solid component by centrifuging the washing solution dispersing the solid component and removing a supernatant after the centrifugation to recover the washed solid component, and the washing solution is a solution selected from the group consisting of a buffer solution with a low ion concentration, a water-soluble organic solvent, water, and a mixture thereof.

(16) The method of recovering a nucleic acid derived from a mammalian cell taken from feces described in the above-mentioned (1), wherein, the fecal sample is agitated at least once in the step (A).

(17) The method of recovering a nucleic acid derived from a mammalian cell taken from feces described in the above-mentioned (1), wherein, the feces is immersed in the treatment solution with a high salt concentration for 12 hours or longer.

(18) The method of recovering a nucleic acid derived from a mammalian cell taken from feces described in the above-mentioned (17), wherein, the feces is immersed in the treatment solution with a high salt concentration for 24 hours or longer.

(19) The method of recovering a nucleic acid derived from a mammalian cell taken from feces described in the above-mentioned (18), wherein, the feces is immersed in the treatment solution with a high salt concentration for 72 hours or longer.

(20) The method of recovering a nucleic acid derived from a mammalian cell taken from feces described in the above-mentioned (1), wherein, the immersing of the faces in the treatment solution is performed at 4° C. or higher.

(21) The method of recovering a nucleic acid derived from a mammalian cell taken from feces described in the above-mentioned (20), wherein, the immersing of the faces in the treatment solution is performed at 10° C. or higher.

(22) The method of recovering a nucleic acid derived from a mammalian cell taken from feces described in the above-mentioned (21), wherein, the immersing of the faces in the treatment solution is performed at 16° C. or higher.

(23) The method of recovering a nucleic acid derived from a mammalian cell taken from feces described in the above-mentioned (1), wherein, 1 volume of the feces is mixed with 1 volume or more of the treatment solution with a high salt concentration in the step (A).

(24) The method of recovering a nucleic acid derived from a mammalian cell taken from feces described in the above-mentioned (1), wherein, the treatment solution with a high salt concentration includes a surface-activating agent.

(25) The method of recovering a nucleic acid derived from a mammalian cell taken from feces described in the above-mentioned (1), wherein, the treatment solution with a high salt concentration includes a coloring agent.

(26) The method of recovering a nucleic acid derived from a mammalian cell taken from feces described in the above-mentioned (1), wherein, the nucleic acid derived from a mammalian cell and the nucleic acid derived from an enterobacterial cell are recovered concurrently from the solid component.

(27) The method of recovering a nucleic acid derived from a mammalian cell taken from feces described in the above-mentioned (1), wherein, the step of recovering the nucleic acid from the solid component in the step (C) further comprises the steps of; (a) eluting, in which the nucleic acid is eluted from the enterobacterial cell and the mammalian cell in the solid component by denaturing a protein in the solid component; and (b) recovering eluted nucleic acid, in which the nucleic acid eluted in the step (a) is recovered.

(28) The method of recovering a nucleic acid derived from a mammalian cell taken from feces described in the above-mentioned (27) further comprising the step of; (c) removing protein, in which the protein denatured in the step (a) is removed after the step (a) and before the step (b).

(29) The method of recovering a nucleic acid derived from a mammalian cell taken from feces described in the above-mentioned (27), wherein, the denaturing of the protein in the step (a) is performed by using one or more selected from the group consisting a chaotropic salt, an organic solvent, and a surface-activating agent.

(30) The method of recovering a nucleic acid derived from a mammalian cell taken from feces described in the above-mentioned (29), wherein, the chaotropic salt is a guanidine salt.

(31) The method of recovering a nucleic acid derived from a mammalian cell taken from feces described in the above-mentioned (28), wherein, the removing of protein in the step (c) is performed by using chloroform.

(32) The method of recovering a nucleic acid derived from a mammalian cell taken from feces described in the above-mentioned (27), wherein, the step (b) for recovering the eluted nucleic acid comprises the steps of: (b1) adsorbing nucleic acid, in which the nucleic acid eluted in the step (a) is absorbed to an inorganic support body; and (b2) eluting absorbed nucleic acid, in which the nucleic acid absorbed in the step (b1) is eluted from the inorganic support body.

(33) A nucleic acid recovered from feces by using the method of recovering a nucleic acid derived from a mammalian cell described in the above-mentioned (1).

(34) A method of analyzing a nucleic acid derived from a mammalian cell using the nucleic acid recovered by the method of recovering a nucleic acid derived from a mammalian cell described in the above-mentioned (1).

(35) The method of analyzing a nucleic acid derived from a mammalian cell described in the above-mentioned (34) wherein the mammalian cell is a gastrointestinal cell.

(36) The method of analyzing a nucleic acid derived from a mammalian cell described in the above-mentioned (34) wherein the mammalian cell is a colonic exfoliated cell.

(37) The method of analyzing a nucleic acid derived from a mammalian cell described in the above-mentioned (34) wherein the nucleic acid derived from a mammalian cell is a marker of neoplastic conversion.

(38) The method of analyzing a nucleic acid derived from a mammalian cell described in the above-mentioned (34) wherein the nucleic acid derived from a mammalian cell is a marker of inflammatory gastrointestinal disease.

(39) The method of analyzing a nucleic acid derived from a mammalian cell described in the above-mentioned (34) wherein the nucleic acid derived from a mammalian cell is a nucleic acid derived from the COX2 (cyclooxygenase-2) gene.

(40) The method of analyzing a nucleic acid derived from a mammalian cell described in the above-mentioned (34) wherein one or more of analyses selected from the group consisting of the expression analysis of mRNA, the mutational analysis of the K-ras gene, and, the DNA methylation analysis are performed.

(41) The method of analyzing a nucleic acid derived from a mammalian cell described in the above-mentioned (40), wherein a portion of a total RNA recovered from the feces is reverse transcribed to convert the total RNA to cDNA, a nucleic acid amplification reaction is performed using the cDNA as a template for, and a product of the nucleic acid amplification reaction is analyzed in the expression analysis of mRNA.

(42) A kit for feces sampling comprising: a treatment solution having high salt concentration; and a feces sampling container holding the treatment solution having high salt concentration.

(43) The kit for feces sampling described in the above-mentioned (42) further comprising: a washing solution.

(44) A fecal sample processing apparatus comprising:

a solution removing device that removes the treatment solution having high salt concentration from the feces sample that stored for a predetermined period by immersing the sampled feces in the treatment solution having high salt concentration.

### BRIEF DESCRIPTION OF THE DRAWINGS

FIG. 1 is a diagram illustrating a fecal sample processing apparatus as an embodiment of the present invention.

FIG. 2 is a diagram illustrating a feces sampling container as an embodiment of the present invention. The feces sampling container can be used for a kit for feces sampling, which is an aspect of the present invention.

FIG. 3A to 3E are diagrams illustrating feces sampling containers as embodiments of the present invention. The feces sampling container can be used in a kit for feces sampling, which is an aspect of the present invention.

FIG. 4 is an image of a stained gel. The gel was obtained by performing electrophoresis. In the electrophoresis, the RNAs recovered from fecal samples in Example 1 were used as the samples.

FIG. 5 is a graph showing calculated results of the expression level of the human COX2 gene in the RNA samples recovered from each fecal sample in Example 1.

FIG. 6 is a graph showing band intensity of rRNAs derived from human and bacterial cells. The rRNA bands were obtained by staining the gel after running electrophoresis using the RNAs recovered from each fecal sample in Example 2 as the samples.

FIG. 7 is an image of a stained gel. The gel was obtained by performing electrophoresis. In the electrophoresis, the RNAs recovered from fecal samples in Example 4 were used as the samples.

FIG. 8. is a graph showing calculated results of the expression level of the 16S rRNA gene in the RNA samples recovered from each fecal samples in Example 4.

### DETAILED DESCRIPTION OF THE INVENTION

[Method of Recovering a Nucleic Acid Derived from Mammalian Cells]

This method of recovering a nucleic acid derived from mammalian cells is an aspect of the present invention (hereinafter, may be referred as “the method of recovering of the present invention”). In this method, feces is collected and mixed with a treatment solution with a high salt concentration. Then, the feces is immersed in the treatment solution for a predetermined period of time. In the feces, there are more of bacteria, such as enterobacteria, than the cells derived from the mammal that excreted it. However, the nucleic acid of mammalian cell can be recovered more selectively than from an enterobacterial cell by recovering a nucleic acid from the feces immersed in the treatment solution with a high salt concentration for a predetermined period of time. The feces is immersed in the treatment solution in a condition where the feces and the solution are mixed (suspended). In other words, a nucleic acid preparation of a mammalian cell with a high purity can be easily recovered. In the preparation, the content of nucleic acid derived from bacterial cells is reduced.

It is not clear why the nucleic acid of a mammalian cell is recovered selectively by immersing the feces in the treatment solution with a high salt concentration in this method of recovering of the present invention (hereinafter may be referred as “selective recovery effect”). One interpretation of this effect is that degradation of a nucleic acid derived from a mammalian cell is suppressed in the high salt concentration condition, while degradation of a nucleic acid derived from bacteria is not inhibited. In other words, the nucleic acid derived from bacteria is degraded faster than that derived from a mammalian cell by storing the feces in the high salt concentration condition. As a result, the nucleic acid derived from a mammalian cell is recovered selectively from the feces after immersing in the treatment solution with a high salt concentration.

The method of recovering of the present invention includes a step (A). In the step (A), the feces is added into the treatment solution with a high salt concentration to prepare a fecal sample. Then, the feces is immersed in the treatment solution with a high salt concentration in the fecal sample for a predetermined period of time.

The treatment solution with a high salt concentration used in the method of recovering of the present invention is a solution in which a salt is dissolved in water or a hydrophilic organic solvent as an active component. The wording “as an active component” means a sufficient amount of salt is included in the treatment solution to obtain the selective recovery effect of the present invention. In a case where the amount of salt is not sufficient, the selective recovery effect of the present invention cannot be obtained.

The salt included in the treatment solution with a high salt concentration as an active component can be selected appropriately from salts which are commonly used in preparation or analysis of a biological sample. For example, it may be hydrochloride salt, sulfate salt, and acetate salt. Also, as an active component, one kind of salt or a combination of two or more salts can be used. It is preferable that the active component in the treatment solution include one or more selected from the group consisting of sodium chloride, potassium chloride, ammonium sulfate, ammonium bisulfate, ammonium chloride, ammonium acetate, cesium sulfate, cadmium sulfate, cesium iron(II) sulfate, chromium(III) sulfate, cobalt(II) sulfate, copper(II) sulfate, lithium chloride, lithium acetate, lithium sulfate, magnesium sulfate, manganese sulfate, sodium sulfide, sodium acetate, sodium sulfate, zinc chloride, zinc acetate, and zinc sulfate. In terms of availability, easiness of the handling, safety, and the like, it is preferable the treatment solution is a solution including sodium chloride and/or ammonium sulfate.

Particularly, sodium chloride is the safest compound among them and can be handled easily in a household. Thus, sodium chloride is particularly useful in the screening examination, such as regular checkups.

The concentration of the salt contained in the treatment solution as an active component (hereinafter may be referred as “the salt concentration”) is not specifically limited as long as the concentration is high enough to obtain the selective recovery effect of the present invention. Therefore, the salt concentration can be set appropriately in view of the kind of salts and solvents and the like. The saturation concentration of each salt is the upper limit of the salt concentration. The lower limit of the salt concentration can be determined by a person having ordinary skill in the art empirically in advance, even though it differs depending on the kind of the salts.

For example, the lower limit of the salt concentration can be determined as explained below. First, a series of salt solutions having different salt concentrations under the saturation concentration are prepared. Then, the feces is immersed in each salt solution for a predetermined period of time to prepare fecal samples. Then, nucleic acids are recovered from the samples. After the recovering the nucleic acids, the nucleic acid derived from a mammalian cell is detected. The lower limit of the salt concentration is the salt concentration at which the detection efficiency of the nucleic acid derived from a mammalian cell is higher than the detection efficiency from a fecal sample that is not treated with a salt solution. The detection efficiency can be determined from the amount of amplified products after amplification reaction of a specific gene derived from a mammal cell. Alternatively, it can be determined from the amount of a representative nucleic acids derived from a mammalian cell in the recovered nucleic acid preparation without the amplification reaction. The representative nucleic acids includes 28S rRNA, 18S rRNA, and the like. Furthermore, as a simulated fecal sample, a mixed liquid containing culture medium of a mammalian cell line and that of bacteria can be used.

In this method of recovering of the present invention, it is preferable to set the salt concentration in the treatment solution to be one half of the saturation concentration of the specific salt or higher, irrespective of the type of salt. More preferably, it is four-fifths of the saturation concentration or higher. Even more preferably, it is almost the saturation concentration of the salt. Even more preferably, it is set to be the saturation concentration of the salt. Because of the sufficiently high salt concentration of the treatment solution, components in the treatment solution seep into the feces quickly, when the feces and the treatment solution are mixed. As a result, nucleic acids derived from a mammalian cell and a bacterial cell can be treated quickly. Furthermore, the higher the concentration of the salt, the more effectiveness can be achieved even if a small amount of the treatment solution with a high salt concentration is used with a feces containing a larger amount of moisture. The salt solution having one half concentration of the saturation concentration or higher, or the salt solution having four-fifths of the saturation concentration or higher can be prepared appropriately by diluting the saturated salt solution with a solvent as in conventional methods.

For example, in a case where sodium chloride is used as the active component in the treatment solution, it is preferable for the salt concentration to be 13% or higher. More preferably, the salt concentration is 20% or higher. Even more preferably, the salt concentration is 26% or higher. Particularly, even more preferably, the salt concentration is between 26% and its saturation concentration. In a case where ammonium sulfate is used as the active component in the treatment solution, it is preferable for the salt concentration to be 20% or higher. More preferably, the salt concentration is 30% or higher. Even more preferably, it is set to be 46% or higher.

In the present invention and this specification of the present invention, the unit “%” in the concentration of salt means “weight %” and calculated by dividing the weight of the solution by that of the solute.

The treatment solution used in the method of recovery of the present invention is a solution in which a salt is dissolved in water or a hydrophilic organic solvent. Biological samples such as feces or the like contain a large amount of moisture in general. Thus, by using water or the hydrophilic organic solvent as a solvent of the treatment solution, it mixes (blends) quickly with the feces. As a result, a higher degree of the selective recovery effect of the present invention can be achieved.

As the hydrophilic organic solvent, a hydrophilic organic solvent having a high solubility to water, a hydrophilic organic solvent that can be blended with water in any ratio, or the like can be named as examples. A preferable hydrophilic organic solvent blended with water has 12 wt % or higher of the solubility against water. More preferably, the solubility is 20 wt % or higher. Even more preferably, it is 90 wt % or more. Particularly, even more preferably, the hydrophilic organic solvent can be blended with water at any ratio. As examples of the hydrophilic organic solvent blended with water at any ratio, methanol, ethanol, n-propanol, 2-propanol, acetone, and the like can be named.

In terms of availability, easiness of handling, safety, and the like, it is preferable the solvent of the treatment solution in this method is water or a water-soluble alcohol. More preferably, it is water, ethanol, propanol, or methanol. Even more preferably, it is water. Furthermore, the solvent can be a mixed solvent made of the solvents described above. For example, the solvent can be a mixed solution including 2 or more solutions with a high salt concentration. Furthermore, it can be a mixed solution made of a solution with a high salt concentration and a different kind of a hydrophilic organic solvent.

The value of pH of the treatment solution with a high salt concentration is not particularly limited as long as degradation of bacterial nucleic acids is not inhibited and degradation of the nucleic acid derived from a mammalian cell in the feces is reduced at the pH value in the salt concentration condition. Preferably, the pH value of the treatment solution is between 4 to 8.

Also, it is preferable that the treatment solution having a high salt concentration has a buffering action. With the buffering action, deviation of the pH value is reduced and kept within the above-mentioned range even if acidic or basic compound, or especially the feces, is added. As a treatment solution having a high salt concentration with a buffering action, an appropriate buffer solution dissolving a salt as the active component can be used. However, in this method of recovery of the present invention, it is particularly preferable that the treatment solution contains an organic acid and its conjugate base. The buffering action can be obtained by containing the organic acid and its conjugate base. For example, the pH value of the treatment solution can be adjusted to an intended value by adding an organic acid and a salt, which is made of the organic acid and an alkaline metal. Alternatively, an organic acid and a salt, which is made of the organic acid and an alkaline earth metal, can be added. As another alternative, a hydroxide of an alkaline metal or an alkaline earth metal can be added after addition of an organic acid to adjust the pH value of the treatment solution.

The treatment solution used in this method of recovery of the present invention may include any other components in addition to the salt, as long as the components do not deteriorate the selective recovery effect of the present invention. For example, a chelating agent which chelates divalent metal ions, a chaotropic salt, and/or a surface-activating agent may be included in the treatment solution. With the chaotropic agent and the surface-activation agent, the cell activity in the feces and the enzyme activity of degrading enzymes can be more effectively inhibited.

The chelating agent that chelates divalent metal ions can be selected from the group indicated below. The group consists of ethylenediaminetetraacetate (EDTA), O, O′-bis(2-amino-phenyl ethylene glycol), ethylenediaminetetraacetate (BAPTA), N,N-bis (2-hydroxyethyl)glycine (Bicine), trans-1,2-Diaminocyclohexane-ethylenediaminetetraacetic acid (CyDTA), 1,3-diamino-2-hydroxypropane-ethylenediaminetetraacetate (DPTA-OH), diethylenetriaminepentaacetate (DTPA), ethylendiamine-dipropanate chloride (EDDP), ethylenediamine-dimethylene sulfate mono hydrate (EDDPO), N-(2-hydroxyethyl)ethylenediaminetriacetate (EDTA-OH), ethylenediaminetetramethylenesulfate (EDTPO), O, O′-bis(2-aminoethyl) ethyleneglycoltetraacetate (EGTA), N,N-bis(2-hydroxybenzyl) ethylenediaminediacetate (HBED), 1,6-hexamethylenediaminetetraacetate (HDTA), N-(2-hydroxyethyl)iminodiacetate (HIDA), iminodiacetate (IDA), 1,2-diaminopropanetetraacetate (Methyl-EDTA), nitrilotriacetate (NTA), nitrilotripropanate (NTP), nitrilotrimethylenesulfate, trisodium salt (NTPO), ethylenediaminetetra(2-pyridylmethyl) (TPEN), triethylenetetraminehexaacetate (TTHA) and the like. Among them, EDTA is preferable. The concentration of the chelating agent is not particularly limited as long as the selective recovery effect of the present invention can be obtained at the concentration. As such, the concentration of the chelating agent can be appropriately set in view of the amount of the feces, steps performed afterward, methods of analyzing the nucleic acid, or the like.

As the chaotropic salt included in the treatment solution of this method of recovery of the present invention, for example, guanidine hydrochloride, guanidine isothiocyanate, sodium iodine, sodium perchlorate, sodium trichloroacetic acid, and the like can be named. As the surface-activating agent included in the treatment solution, non-ionic surface-activating agents are preferable. As the non-ionic surface-activating agent, for example, Tween 80, CHAPS (3-(3-cholamidepropyldimethylammonio)-1-propanesulphonate), Triton X-100, Tween 20, and the like are named. The concentrations of the chaotropic agent and the surface-activating agent are not particularly limited as long as the selective recovery effect of the present invention can be obtained at the concentrations. As such, the concentration of the chelating agent can be appropriately set in view of the amount of the feces, steps performed afterward, methods of analyzing the nucleic acid, or the like.

In addition thereto, a coloring agent can be added appropriately to the treatment solution. By coloring the treatment solution with a high salt concentration, accidental ingestion can be prevented. Also, the color of the feces can be weakened. A preferable coloring agent is one used as an additive for foods. The preferable color is blue, green, or the like. Specific examples can be the Fast Green FCF (Green No. 3), Brilliant Blue FCF (Blue No. 1), Indigo Carmine (Blue No. 2), and the like can be named. One coloring agent can be used alone, or two or more coloring agents can be used together.

The volume of the treatment solution with a high salt concentration is not particularly limited. However, the mixing rate of the feces and the treatment solution is 1 volumetric part of the feces for 1 or more volumetric parts of the treatment solution. When the feces is inserted into a feces sampling container, the whole outer surface of the feces could be immersed completely in the treatment solution, if there were the treatment solution, volume of which equals that of the feces or more. As a result, the selective recovery effect of the present invention can be obtained efficiently. For example, in a case where the volume of the feces and that of the treatment solution are the same, it is possible to decrease the weight or to downsize the feces sampling container. On the other front, by adding the treatment solution, the volume of which is five times or more than that of the feces, dispersion of the feces in the treatment solution can be done quickly and effectively. In addition, decreasing effect of the salt concentration due to the diluting effect by the moisture included in the feces can be circumvented. To be benefited by having the feces sample container to be light-weighted and downsized, and by improved dispersion of the feces at the same time, it is preferable that the mixing ratio between the feces and the treatment solution be 1:1 to 1:20 in volume. More preferably, the ratio is 1:3 to 1:10. Even more preferably, it is substantially 1:5.

The feces subjected to the method of recovering of the present invention is not particularly limited as long as it is of an animal. Preferably, the feces is taken from a mammalian animal. More preferably, it is taken from a human. For example, it is preferable that the feces is those taken from a human collected in the regular checkups or a diagnosis of a visiting patient. However, it could be taken from a farm or wild animal. The feces that is preserved for a certain period of time after collection could be used in this method of recovery, even though it is preferable that the feces is processed immediately after collection. In addition, the feces that is left for a certain period of time after its egestion can be used, even though it is preferable that the feces is processed immediately after its egestion.

The amount of the feces subjected to the method of recovery of the present invention is not particularly limited. A preferable amount of the feces is 10 mg to 1 g. If too much feces is used in this method of recovery, collecting operation takes too long, and a larger size of the feces sample container is needed. As a result, handling the feces and the container will be cumbersome. On the other hand, if too little feces is used, the number of the mammalian cells, such as colonic exfoliated cells, in the feces becomes too small. As a result, sufficient amount of nucleic acids cannot be obtained, and the accuracy of the intended nucleic acid analysis will be deteriorated. The feces is heterogeneous, and contains varieties of components that are unevenly distributed within. Therefore, it is preferable to collect the sample from a broad area on the specimen of the feces.

In the preparation of fecal sample in the step (A), what is needed is adding the treatment solution with a high salt concentration to the feces and immersing the feces in the treatment solution. Thus, it is not essential to perform a particular agitating (stirring) operation. The treatment solution with a high salt concentration used in the method of recovering of the present invention is mixed extremely well to biological sample, such as feces or the like including a large amount of moisture. Therefore, depending on the amount and state of the feces added to the treatment solution, the treatment solution could seep in to the biological sample. In such a case, when the feces is just immersed in the solution without the agitating (stirring) operation, the selective recovery effect of the present invention can be obtained.

Also, it is preferable to perform agitating (stirring) and blending after adding the treatment solution to the feces. By agitating (stirring), the feces is sufficiently dispersed in the treatment solution with a high salt concentration. As a result, the feces is suspended in the solution. In a case where the agitating (stirring) of the feces in the treatment solution is preformed, the agitating (stirring) can be performed at any time in the step (A). As such, the agitating (stirring) may be performed immediately after the addition of the solution to the feces, after immersing in the solution for a predetermined period of time, or during the immersion appropriately. In the method of recovery of the present invention, it is preferable that the agitating (stirring) is performed after the addition of the solution to the feces quickly. By dispersing the feces in the treatment solution quickly, the salt, which is the active component, can quickly seep into the cells in the feces. As a result, the selective recovery effect can be achieved at even higher level. In the method of recovery of the present invention, it is preferable to perform the agitation (stirring) at least once, after the preparing the fecal sample by adding the treatment solution to the feces and before recovering a solid component from the fecal sample in the step (B).

The methods of agitating and blending the feces and the treatment solution are not particularly limited as long as they are physical techniques. For example, a collected feces is inserted in a container that can be sealed air-tight holding the treatment solution. After putting the feces in the container, it is sealed. Then, the contents in the container may be blended by vertically inverting the container. Alternatively, the contents may be blended with a shaker, such as a vortex mixer or the like. Also, they may be blended in the presence of blending particles. The method of using a shaker or blending particles is preferable since the contents of the container can be blended quickly. In particular, by using a feces sampling container including the blending particles in advance, the feces and the treatment solution in the container can be blended quickly even in an environment without special equipment, such as a common household or the like.

The blending particles are not particularly limited as long as they do not deteriorate the selective recovery effect of the present invention obtained by the treatment solution. Also, it is needed that they are the particles with a hardness and a specific density allowing to disperse the feces quickly and sufficiently by colliding with the feces. The blending particles can be a population of grains with one composition. Alternatively, they can be a population of grains having two or more different compositions. As examples of the blending particles, particles made of glass, ceramic, plastic, latex, metal, and the like can be named. On the other front, magnetic blending particles or non-magnetic blending particles may be used.

The selective recovery effect of the present invention can be simply immersing the feces in the treatment solution with a high salt concentration after adding the solution to the feces. Thus, it not essential to blend the feces and the treatment solution immediately after the addition of the solution to the feces. Accordingly, they may be blended by vibration during transportation to the storage place.

As described above, the fecal sample is obtained by adding the treatment solution with a high salt concentration to the feces. By preserving the fecal sample for a predetermined period of time, the feces is immersed in the treatment solution. The amount of time the feces is immersed in the solution is not particularly limited as long as it is long enough to obtain the selective recovery effect of the present invention. As such, the amount of time can be set in consideration of the type and the concentration of the salt which is the active component of the solution, the mixing ratio between the feces and the treatment solution, the preserving temperature, and the like. In the method of recovery of the present invention, it is preferable to immerse the feces for more than an hour. More preferably, it is immersed for more than 12 hours. Even more preferably, for more than 72 hours. Also, the feces can be immersed in the treatment solution for more than 168 hours. For example, in a case where the feces is immersed in the solution for more than an hour, the treatment solution with a high salt concentration is seeped in sufficiently to the whole body of the feces irrespective of the condition of the collected feces. As a result, degradation of the nucleic acids derived from mammalian cells is suppressed, while a sufficient amount of the nucleic acids derived from bacterial cells is degraded. Accordingly, the selective recovery effect on the nucleic acids derived from mammalian cells can be achieved at a high level.

The selective recovery effect of the present invention, which can be achieved by using the treatment solution with a high salt concentration, is intensified by performing the immersion at a high temperature condition rather than at a low temperature condition. Specifically, it is preferable to perform the immersion in the step (A) at 4° C. or higher. More preferably, it is performed at 10° C. or higher. Even more preferably, at 16° C. or higher. Above all, the temperature for the preserving is preferably 50° C. or lower.

In the method of recovering of the present invention, as long as the immersing of the feces is performed at 4° C. or higher, the selective recovery effect of the present invention can be achieved. As such, the immersing treatment in the step (A) may be performed under a condition where the temperature is controlled with a constant-temperature unit or the like. Alternatively, it can be performed at room temperature in an environment without controlling the temperature. In addition, the immersing can be performed at the temperature, at which general feces sampling, transportation of the feces, or the like is performed. Therefore, in a case where the fecal sample is transported under a temperature-uncontrolled condition, the time of the period that the fecal sample is transported functions as the immersion treatment period (the period that the feces is immersed in the treatment solution). As a specific example, a case described below can be conceived. In the example, the location where the feces sample is prepared by a sampler and the location where that the nucleic acid analysis is performed are far apart. The prepared fecal sample is transported from the first location to the second location. As long as the temperature of the fecal samples during the transportation being kept between 4 to 50° C., the transporting time works as the immersing treatment period with or without of controlling of the temperature during the transportation.

As explained above, nucleic acids in the feces are liable to be degraded. Therefore, fecal samples are usually subjected to the steps for recovering and analyzing nucleic acids immediately after the preparation of the fecal samples. Also, in a case where there is long time lag between the preparation of the fecal samples and the recovery and analyzing of the nucleic acids, the fecal samples are stored in a low temperature condition, such as by freezing or refrigeration to suppress the degradation of the nucleic acids. Contrary to that, in the method of recovery of the present invention, a nucleic acid preparation with a high quality can be recovered efficiently even if the fecal samples are stored at a relatively high temperature, such as room temperature, for a long period of time. In the high quality nucleic acid preparation, there are less contaminating nucleic acids derived from bacterial cells.

After the step (A) explained above, solid components are recovered from the fecal sample as the step (B). Then, after the step (B), nucleic acids are recovered from the solid components as the step (C).

The method of recovering a solid component from the fecal sample in the step (B) is not particularly limited. What is essential for the method is that the treatment solution, which is the liquid component of the fecal sample, can be separated from the solid component without damaging the nucleic acids derived from mammalian cells in the fecal sample in the method. Thus, the method can be chosen appropriately from conventional separating methods, in which liquid and solid components are separated. For example, the recovery of the solid component may be performed by centrifugation, in which the solid component derived from the feces is recovered as a precipitate by removing a supernatant after centrifugation. Alternatively, the recovery of the solid component may be performed by filtration, in which the solid component left on a filter surface is recovered after filtering the fecal sample.

In the solid component recovered in the step (B), there is a large amount of residual salts originated from the treatment solution with a high salt concentration. Therefore, in a case where the nucleic acids are prepared from the solid component containing a large amount of salts directly, the final nucleic acid preparation would contain salts at a high concentration. In a case where the nucleic acid preparations contain salt at a high concentration, activities of enzymes, such as polymerase or the like, can be suppressed in a reverse transcription reaction, a nucleic acid amplification reaction, or the like, in the analysis of the nucleic acids. For example, it has been reported that the extension reaction by a polymerase in the polymerase chain reaction (PCR) is inhibited when the reaction solution contains 30 mM or more of magnesium ions, or 100 mM or more of sodium ions (see APPLIED AND ENVIRONMENTAL MICROBIOLOGY, volume 64, issue 10, page 3748-3753). Therefore, it is preferable to reduce the content of salts in the solid component recovered in the step (B) before being subjected to preparation of the nucleic acids by washing the solid component with an appropriate washing solution.

The step (D) washing the solid component can be performed after the step (B) and before the step (C). In the step (D), the solid component obtained in the step (B) is dispersed in a washing solution in a step (d). Then, the washed solid component is recovered by centrifuging the washing solution dispersing the solid component and removing the supernatant after the centrifugation in the step (e). The recovery from the suspension can be performed as in the recovery of the solid component from the fecal sample in the step (B). In the method of recovery of the present invention, it is preferable to recover the solid component from the suspension by removing the supernatant after centrifugation of the suspension. As described above, by having the washing process before the step (C) recovering the nucleic acid, the residual salts can be removed from the solid component efficiently. As a result, the reverse transcription and nucleic acid amplification reaction can be performed efficiently by using the nucleic acid preparations from the washed solid components.

The washing solution is not particularly limited as long as the content of salt can be reduced enough with the solution. As examples of the washing solutions, a buffer solution with a low ion concentration, a hydrophilic organic solvent, water, or a mixture thereof can be named. As examples of the buffer solution with a low ion concentration, the phosphate buffer, Tris buffer, or the like, which are generally used in this technical field, can be named. A preferable concentration of the buffer solution with a low ion concentration is less than 1 M. A more preferable concentration of the buffer solution is less than 0.3 M. An even more preferable concentration is less than 0.1 M. In a case where the ion is magnesium or the like, the preferable concentration is less than 0.03 M particularly. As the hydrophilic organic solvent, the solvents applicable to the solvents of the treatment solution with a high salt concentration can be used similarly. In the method of recovery of the present invention, it is particularly preferable to use an acidic solution of an organic solvent with a low ion concentration. A reason for the advantage using the acidic solution is that by washing the solid component under an acidic condition, hydrolysis of the nucleic acids in the solid component during the washing process can be effectively suppressed.

In the step (D) washing the solid component, it is preferable to wash the solid component until the concentration of salts in the solid component is reduced to less than 100 mM. If the concentration of salt in the solid component before extraction of nucleic acids was reduced to less than 100 mM, the amount of salt brought into the nucleic acid preparation, which is extracted and recovered from the solid component, could be sufficiently reduced. As a result, the reverse transcription reaction and the nucleic acid amplification reaction are not interfered even if the nucleic acid preparation is directly used for the reactions without performing a specific desalting treatment.

The method of recovering a nucleic acid from the solid component in the step (C) is not particularly limited. As long as it is one of the conventional methods of recovering nucleic acids from a sample, any method can be chosen. The nucleic acids recovered from the solid component derived from the feces may be DNA, RNA, or a mixture thereof. In the method of recovery of the present invention, recovering RNA is particularly preferable. For example, in the step (C), a step (a) can be performed. In the step (a), nucleic acids are eluted from mammalian cells and enterobacterial cells in the solid component, which is recovered in the step (B) or recovered after washing of the solid component (hereinafter may be referred as “the solid content derived from feces”). In the step (a), proteins in the solid content derived from feces are denatured. After the step (a), a step (b) is performed. In the step (b), the eluted nucleic acids are recovered. As explained above, by performing the steps (a) and (b) in the step (C), nucleic acids can be recovered from the solid content derived from feces.

The denaturation of proteins in the solid content derived from feces in the step (a) can be performed by conventional denaturing methods. For example, by adding a chemical commonly used as a protein denaturant, such as a chaotropic agent salt, an organic solvent, a surface-activating agent, or the like, the proteins in the solid content derived from feces can be denatured. The chaotropic agent salts and the surface-activating agents, which were named as those that can be added to the treatment solution with a high salt concentration, can be used in the step (a). As an organic solvent, phenol is preferable. The phenol may be neutral or acidic. In a case where the acidic phenol is used, RNA is extracted to an aqueous phase more selectively than DNA. In a case where a chaotropic agent, an organic solvent, a surface-activating agent, or the like is added to the solid component derived from feces, only one type of chemical may be added. Alternatively, two or more chemicals may be added.

In the denaturation, it is preferable to add the protein denaturants after suspending the solid content derived from feces in an elution reagent once, even though the protein denaturants can be added directly to the solid content. In a case where DNA is recovered, the phosphate buffer, Tris buffer, or the like can be used as the elution reagent. It is preferable that the DNase in the elution reagent is inactivated by autoclaving or the like. In addition, it is preferable that the elution reagent contains a protease, such as the proteinase K. In a case where RNA is recovered, the citrate buffer or the like can be used. However, it is preferable to use a buffer containing an RNase inhibitor, such as guanidine thiocyanate, guanidine hydrochloride, or the like, since RNAs are easily degraded.

After the step (a) and before the step (b), as a step (c) the denatured proteins may be removed. The quality of the nucleic acid preparation is improved by removing the denatured proteins in advance before recovery of the nucleic acids. The removal of the denatured proteins in the step (c) can be performed by conventional methods. For example, the denatured proteins can be removed by recovering the supernatant alone after sedimenting the denatured proteins by centrifugation. In addition, the denatured proteins can be removed more thoroughly than the case explained above by the procedure explained below. In this procedure, chloroform is added after the step (a) and mixed well with a vortex mixer or the like. Then, the mixture is subjected to centrifugation to sediment the denature proteins. After the centrifugation, the supernatant in which the denature proteins are removed is recovered.

The recovery of nucleic acids in the step (b) can be performed by conventional methods such as ethanol precipitation, cesium chloride ultra centrifugation. Alternatively, the nucleic acids can be recovered by performing a step (b1) and (b2) which are explained below. In the step (b1), the eluted nucleic acids obtained in the step (a) are adsorbed to an inorganic support. After the step (b1), the adsorbed nucleic acids are eluted from the inorganic support. As the inorganic support used in the step (b1), conventional inorganic supports capable of adsorbing nucleic acids can be used. The shape of the inorganic support is not particularly limited, and it can be fine particles or membrane-shaped. As examples of the inorganic support, particles containing silica (beads), such as a silica gel, a siliceous oxide, glass, diatom earth, or the like can be named. In addition, porous membranes such as nylon, polycarbonate, polyacrylate, nitrocellulose, or the like can be named. The solvent used in the step (b2) for eluting the nucleic acids adsorbed to the inorganic support can be appropriately selected from conventional solvent commonly used for eluting nucleic acids from an inorganic support, in view of the type of the nucleic acids, the method of analyzing the nucleic acids afterward, or the like. As the solvent for eluting the adsorbed nucleic acid from the inorganic support, the purified water is particularly preferable. Furthermore, it is preferable to wash the inorganic support retaining the nucleic acids after the step (b1) and before the step (b2) with an appropriate washing buffer.

Depending on the method of analyzing nucleic acids afterward, the nucleic acids can be recovered by eluting from the solid content derived from feces without extraction and purification of nucleic acids. In such a situation, simply an appropriate eluting reagent is added to the solid content, and the reagent and the solid content are mixed. Alternatively, the recovery of nucleic acids from the solid content derived from feces can be done using a commercially available kit, such as a nucleic acids extraction kit or the like.

In the testing of clinical specimens, increasing of the number of steps leads to high labor cost. However, in the recovery method of the present invention, the step, in which the nucleic acids derived from mammalian cells are selectively enriched, can be performed before the testing step in the testing site by a feces collector who immerses the feces in the treatment solution with a high salt concentration directly. Therefore, the method of recovery of the present invention can contribute to cost reduction in clinical testing or the like.

[Method of Analyzing a Nucleic Acid Derived from Mammalian Cells]

In general, in a case where nucleic acids are recovered from feces directly without recovering the mammalian cells, the majority of the recovered nucleic acids is originated from bacteria, such as enterobacteria. However, in the method of recovery of the present invention, degradation of nucleic acids derived from bacterial cells is enhanced while degradation of nucleic acids derived from mammalian cells is suppressed, by immersing the solid content derived from mammalian cells in the treatment solution with a high salt concentration before extraction and recovery of the nucleic acids. As a result, the nucleic acids derived from mammalian cells can be selectively recovered even if the total nucleic acids from all organisms included in the feces are recovered without separating materials by the type of organisms.

As explained above, in the method of recovery of the present invention, a highly pure preparation of nucleic acids, which contains less nucleic acids derived from bacterial cells such as enterobacteria or the like, can be obtained efficiently. Therefore, it is expected that a highly reliable analysis results would be obtained by performing analysis using the nucleic acid preparation recovered by the recovery method of the present invention in the analysis of the nucleic acids derived from mammalian cells, the content of which is relatively low. For example, disease-associated genes (disease markers) such as human oncogenes can be detected with high sensitivity and accuracy by using the nucleic acids preparation recovered in the method of recovery of the present invention

In particular, since the nucleic acids are recovered from the feces, it is preferable that the nucleic acid preparations, which are recovered in the recovery method of the present invention, are subjected to the analysis of nucleic acids derived from gastrointestinal tract cells such as those in the large intestine, the small intestine, the stomach, or the like. It is more preferable to use the nucleic acid preparation for analyzing nucleic acids derived from colonic exfoliated cells. For example, there are cases in which early detection is critical for certain types of cancer. Also, there are cases in which an accurate detection of a cancer is needed. The nucleic acids prepared in the method of recovery of the present invention are suitable for the cases described above. In addition, the nucleic acid preparation obtained by the method of recovering of the present invention is suitable for nucleic acid analysis testing the onset of colitis, inflammation of the small intestine, gastritis, inflammatory diseases such as pancreatitis, or the like. Alternatively, the nucleic acid preparation can be subjected to a test of polypoid lesions such as polyps and diseases of the large intestine, the small intestine, the stomach, the liver, the gallbladder, the bile duct, or the like such as the gastric ulcer.

Particularly, it is preferable to detect a makers indicating neoplastic conversion or a inflammatory gastrointestinal disease. For example, as the maker indicating neoplastic conversion, the conventional cancer markers such as carcinoembryonic antigen (CEA) and sialyl Tn antigen (STN) can be named. Also the presence or absence of mutations on the other known cancer marker gene, APC gene, p53 gene, K-ras gene, or the like can be detected. In addition, it is useful to detect methylation of genes such as p16, hMLHI, MGMT, p14, APC, E-cadherin, ESR1, SFRP2, or the like as a diagnostic marker of colorectal disease (for example, see Lind et al., “A CpG island hypermethylation profile of primary colorectal carcinomas and colon cancer cell lines”, Molecular Cancer, 2004, Chapter 28, Volume 3). On the other hand, as an example of the marker indicating an inflammatory gastrointestinal disease, COX2 (cyclooxygenase-2) or the like is named.

The nucleic acids recovered by the method of recovering of the present invention can be analyzed by conventional nucleic acid analyzing methods. As examples of the analyzing methods, a method of quantifying the nucleic acids can be named. Also a method of detecting a region with a specific base sequence by analyzing an amplified product obtained from a nucleic acid amplification reaction such as PCR can be named. In a case where RNAs are recovered, the RNAs are converted to corresponding cDNAs by reverse transcription. After the conversion, the obtained cDNAs are analyzed in the same way as DNA. In a case where the DNA is recovered from the fecal sample, for example, methylation on DNA and mutations such as insertion of a base, deletion, substitution, duplication, inversion, or like can be detected. In addition, the presence or absence of cancer can be tested by detecting a genetic mutation on a sequence region or the like including the microsatellite. In a case where RNA is recovered, mutations on RNA such as insertion of a base, deletion, substitution, duplication, inversion, splicing variants (isoforms) or like can be detected. In particular, it is preferable to perform an expression analysis of mRNA, a mutational analysis of K-ras gene, DNA methylation analysis, or the like. These analyses can be performed by the conventional methods in this technical field. Also, a commercially available kit for the mutational analysis of K-ras gene or the methylation detection may be used.

In the present specification, enterobacteria means the bacterial cells that exist in feces in a relatively large number and normally inhabits in the gut of an animal such as a human. As examples of the normal inhabitant of the gut, an obligate anaerobic bacteria such as genus Bacteroides, genus Eubacterium, genus Bifidobacterium, genus Clostridium, or the like, and a facultative anaerobic bacteria such as, genus Escherichia, genus Enterobacter, genus Klebsiella, genus Citrobacter, genus Enterococcus, or the like can be named.

### [A Fecal Sample Processing Apparatus]

The step (B) and the washing step, which is performed is it is necessary, in the method of recovery of the present invention can be performed easily and quickly with a processing apparatus. For example, the apparatus includes a solution removing mechanism for removing the treatment solution with a high salt concentration, which is the liquid component, from the fecal samples. The solution removing mechanism is not particularly limited as long as it is a mechanism separating the solid and liquid components. However, it is preferable that the solution removing mechanism is a centrifugation mechanism. Additionally, by including a mechanism for aspirating and draining and a mechanism for collecting an effluent, it would be possible to automate the step (B) for multiple fecal samples. The mechanism for aspirating and draining removes the supernatant that was separated by the centrifugation mechanism. It is preferable that the mechanism for aspirating and draining is a nozzle that aspirates the supernatant from the tip of the nozzle. As examples of such a nozzle, an electronically motorized pipette or the like can be named.

Contamination between fecal samples can be avoided in a processing apparatus having the nozzle for aspirating and draining, if the apparatus further includes a mechanism for washing the nozzle. In the processing apparatus with the washing mechanism, the nozzle can be cleaned for each fecal sample in the process of removing the supernatant from the fecal samples. Alternatively, the contamination can be avoided without the mechanism for washing the nozzle, if the apical end of the nozzle is an exchangeable pipette chip or the like. The processing apparatus needs to further include a device for attaching and detaching the chip automatically to exchange the tips for each fecal sample.

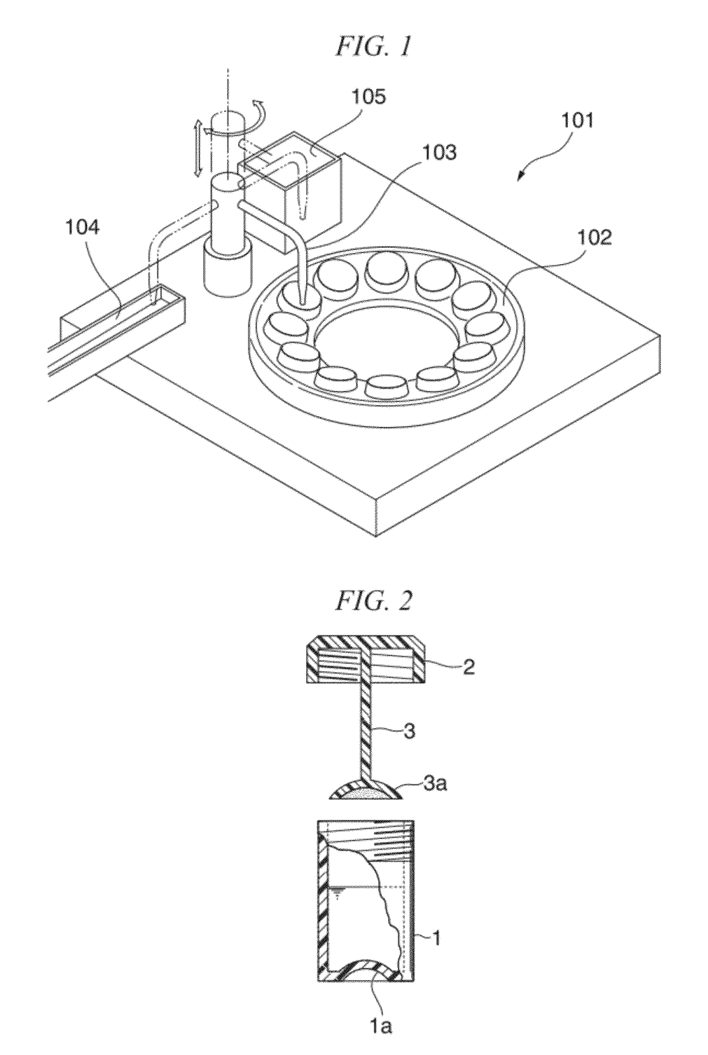

FIG. 1 shows a fecal sample processing apparatus which is an embodiment of the present invention. With the processing apparatus, the step (B) in the method of recovery of the present invention is automated. The processing apparatus 101 of the embodiment of the present invention includes a mechanism for centrifugation 102, a nozzle for aspirating and draining 103, which aspirates and removes the supernatant separated with the mechanism for centrifugation 102, an effluent reservoir 104, and a mechanism 105 for washing the nozzle for aspirating and draining. First, each sample of feces is placed on the mechanism for centrifugation 102 of the fecal sample processing apparatus 101 in a condition where each lid of the fecal sample container is opened. In the fecal sample container, collected feces has been immersed in the treatment solution with a high salt concentration for a predetermined period of time. Then, the fecal samples are centrifuged. After centrifugation, the apical end of the nozzle for aspirating and draining 103 is allowed to contact to the supernatant of a fecal sample. Then, the supernatant is aspirated and removed from the fecal sample container. The supernatant aspirated with the nozzle for aspirating and draining 103 is drained to the effluent reservoir 104. After that, the part of the nozzle that contacted the supernatant is washed by the washing mechanism of the nozzle 105. After the washing, the supernatant is removed from another fecal sample by aspiration in the same manner. As explained above, by using the fecal sample processing apparatus shown in FIG. 1, the step (B) or the like can be automated. In the processing apparatus, the supernatant is removed by aspiration sequentially from multiple samples of feces, which were centrifuged together once.

### [A Kit for Feces Sampling]

It is possible to prepare a fecal sample quickly from a collected feces by collecting the feces in a fecal sample container that was allowed to hold the treatment solution with a high salt concentration in advance. In addition, it is possible to perform the method of recovering of the present invention even more easily with a kit for feces sampling including the treatment solution with a high salt concentration and the fecal sample container holding the treatment solution. In addition to the treatment solution and the container, the kit for feces sampling may appropriately include other components, such as a bar for collecting feces, a washing solution, or the like.

As such, the form, size, or the like of the fecal sample container are not particularly limited, and conventional fecal sample containers that can hold a solvent can be used. It is preferable that the container include a lid combined with the feces collecting bar, since ease of handling would be improved. In addition, it is preferable that the feces collection bar has a form suitable to collect a certain amount of the feces. As an example of a conventional fecal sampling container, the container disclosed in Japanese Examined Patent Application, Second Publication No. H6-72837 or the like can be named.

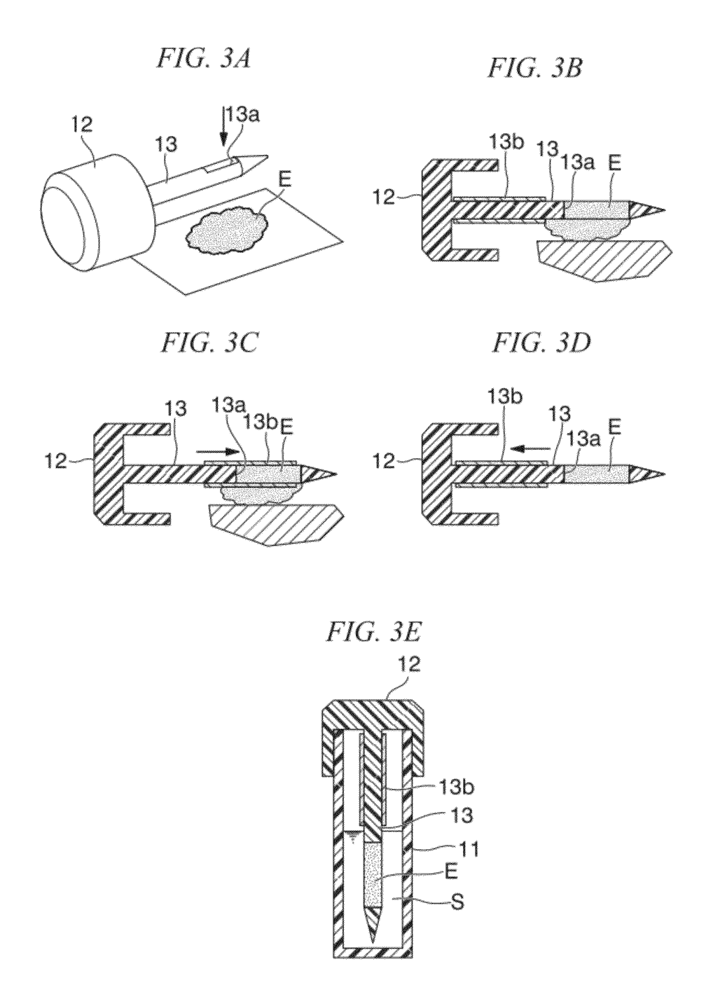

FIGS. 2 and 3 shows an embodiment of the fecal sample container which can be used in the kit for feces sampling, which is an aspect of the present invention. However, the kit for feces sampling that can be used in the aspect of the present invention is not limited to the embodiment.

First, the fecal sample container in FIG. 2 is explained. The fecal sample container includes a lid 2 combined with a bar for collecting feces 3, and a container main body 1. In the container main body 1, the treatment solution with a high salt concentration S is contained. On the apical end of the bar for collecting feces 3, a cup 3a, which holds a certain amount of the feces, is provided. On the cup 3a, multiple sieving holes are formed. By engaging the cup 3a and the bumped portion 1a, the feces in the cup 3a is pressed and squeezed out from the outer openings of the holes. As a result, the feces can be quickly suspended in the treatment solution with a high salt concentration S.

The fecal sample container shown in FIG. 3 includes a lid 12, which is combined with a bar for collecting feces 13 whose apical end is pointed, and a container main body 11. The treatment solution with a high salt concentration S is contained in the container main body 11. On the bar for collecting feces 13, a hole 13a, which can hold a certain amount of the feces E, is formed. In addition, a movable lid 13b, which functions as a lid by sliding on the collecting bar 13, is provided on the bar for collecting feces 13. As shown in the FIG. 3A, the movable lid 13b is moved toward the lid side to keep the opening of the hole 13a to be opened completely. Then, the hole 13a is pressed toward the feces E. As a result, as indicated in FIG. 3B, the hole 13a is filled with the feces E. By sliding the movable lid 13b toward the apical end of the collecting bar 13, the outer openings of the hole 13a are closed. In this way, the exact volume of the feces, which equals to the volume of the vacant space of the hole 13a, can be collected (FIG. 3C). After that, the outer openings of the hole 13a are completely opened by sliding the movable hole 13b toward the lid side again (FIG. 3D). Then, the lid 12 is assembled with the main container body 11 (FIG. 3E). The fecal sample container explained above can be handled safely in a common household.

### EXAMPLES

More details of the present invention are explained below. However, the present invention is not limited by examples described below. In terms of MKN45 cells and the bacterial cells (Enterobacter aerogenes), cells cultivated in conventional methods are used.

### Example 1

By using a simulated fecal sample, the selective recovery of nucleic acids derived from mammalian cells in the method of recovery of the present invention is confirmed.

First, feces were collected from three healthy individuals and suspended in a bacterial culture. Then, a fecal solution was prepared from the supernatant of the suspension. The simulated fecal sample was prepared by mixing the human cancer cell derived MKN45 cells to the fecal solution.

Then, the simulated fecal sample was aliquoted to five 15 mL volume polypropylene tubes, each of the tubes containing 1 g of the simulated fecal sample. Nothing else was added (Fecal sample 1A) to one of the 5 tubes, and it was immediately processed with centrifugation. Then a solid content was recovered by removing the supernatant. RNA was recovered from the solid content of the fecal sample 1A.

Ten milli litter of the following solutions were added to each of the remaining 4 tubes (Fecal sample 1B, 1C, 1D, and 1E) immediately after being aliquoted and sufficiently mixed. The 4-fold dilution of the saturated sodium chloride solution was added to the fecal sample 1B. The 2.7-fold dilution of the saturated sodium chloride solution was added to the fecal sample 1C. The 2-fold dilution of the saturated sodium chloride solution was added to the fecal sample 1D. The saturated sodium chloride solution was added to the fecal sample 1E. Then, these 4 fecal samples were allowed to stand for 24 hours at room temperature. After that, they were immediately centrifuged. After removing the supernatant, the solid contents were recovered from the remaining 4 fecal samples. Sodium acetate buffer (0.1 M) was added to the solid contents as a washing solution. After dispersing the solid contents sufficiently, they were centrifuged again. The washed solid contents were obtained by removing their supernatants.

The concentration of the saturated sodium chloride is 26.38% at 20° C. Therefore, the saturated solution of sodium chloride used in this Example is 26%. Similarly, the concentration of sodium chloride in the 2-, 2.7-, 4-fold dilutions are 13%, 9.75%, and 6.5%, respectively.

Recovery of RNA from the obtained solid contents was performed as described below. First, the phenol mixture “Trizol” (manufactured by Invitrogen Corp.) to the solid content and mixed sufficiently with a homogenizer. Then, chloroform was added and the samples were mixed sufficiently with a vortex mixer. Then, the samples were centrifuged at 12,000×g for 20 min at 4° C. Finally, RNA was recovered from the supernatant (aqueous phase) by the ethanol precipitation method. Specifically, sodium acetate and 100% ethanol was added to the supernatant. After mixing the samples, precipitations were obtained after centrifugation. The precipitates were dried after washing. Solutions of RNA preparation were obtained by dissolving the dried precipitates in DEPC-treated water.

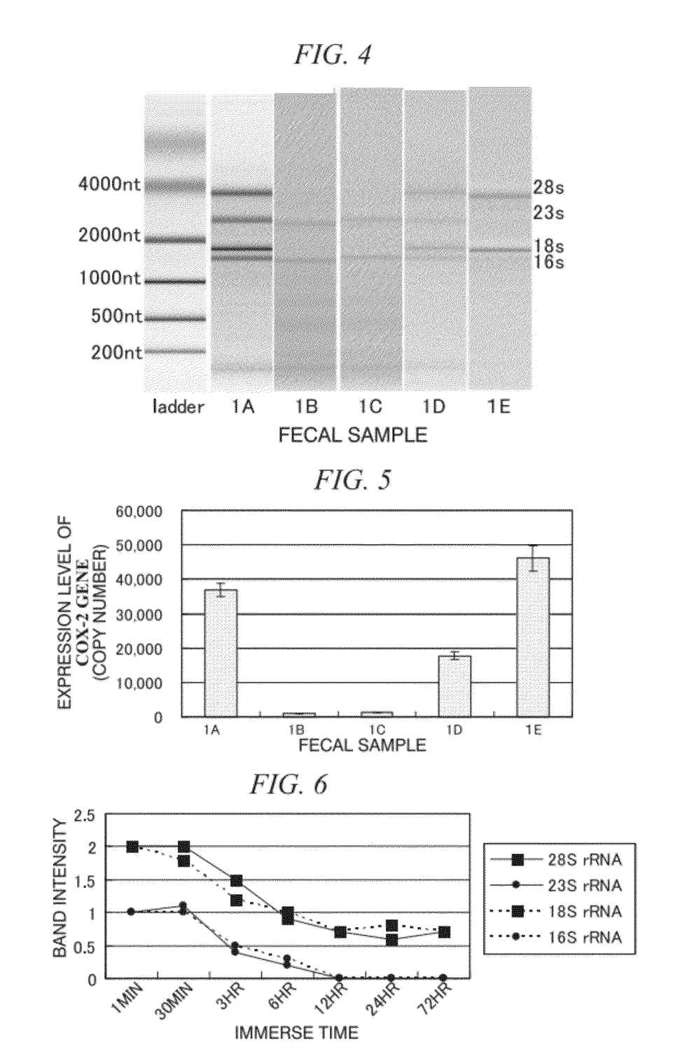

The obtained RNA solutions were subjected electrophoresis. After the electrophoresis, the gel was stained and bands representing RNA populations were visualized. The obtained image of the stained gel is shown in FIG. 4. In FIG. 4, the lane labeled as “ladder” is the lane in which a size maker was run. In TABLE 1, relative band intensity of bands representing 16S rRNA, 23S rRNA, 18S rRNA, and 28S rRNA in the stained image is shown. From the result shown in Table 1, it was confirmed that the higher the concentration of sodium chloride added to the simulated fecal sample, the stronger the band intensity of 18S rRNA and 28S rRNA, which are nucleic acids derived from mammalian cells, while the weaker the band intensity of 16S rRNA and 23S rRNA, which are nucleic acids derived from bacterial cells. In particular, in the fecal sample 1E, in which the saturated solution of sodium chloride was added, only the nucleic acids derived from mammalian cells were detected, while the nucleic acids derived from bacterial cells were not. Contrary to that, in the fecal samples 1B, in which the 4-fold dilution of the saturated sodium chloride solution was added, and 1C, in which the 2.7-fold dilution of the saturated sodium chloride solution was added, only the nucleic acids derived from bacterial cells were detected, while the nucleic acids derived from mammalian cells were not. From the result explained above, it was confirmed that the nucleic acids derived from mammalian cells can be recovered more selectively than the nucleic acids derived from bacterial cells, by immersing feces in a salt solution having sufficiently high salt concentration (treatment solution with a high salt concentration) for a predetermined period of time.

TABLE 1

Band Intensity of rRNA
(Relative Value)

| Fecal Samples | 16S | 18S | 23S | 28S |
|---|---|---|---|---|
| 1A | 68 | 78.5 | 71.7 | 71.3 |
| 1B | 3.1 | 0 | 3.6 | 0 |
| 1C | 3.5 | 0 | 4 | 0 |
| 1D | 5.3 | 7.3 | 4.6 | 7.1 |
| 1E | 5.3 | 13.1 | 4 | 13.5 |

Using the obtained RNAs from the fecal samples as a template, cDNAs were prepared by performing reverse transcription reactions. The amount of RNAs in each reverse transcription was adjusted to 1 μg. Using the obtained cDNAs, a DNA region corresponding to the mRNA of the human COX2 gene was amplified with the TaqMan PCR kit, and the amplified products were detected. As a realtime PCR primer, the COX2 Primer Probe MIX manufactured by Applied Biosystems Corp. (Catalog No. Hs00153133_ml) was used. Specifically, the PCR reaction mixture was prepared as explained below. First, 1 μL, of each cDNA sample was aliquoted to each 0.2 ml volume well of the 96-well PCR plate. After that, 8 μL, of ultrapure water and 10 μL, of the nucleic acid amplification reagent “TaqMan GeneExpression Master Mix” (manufactured by Applied Biosystems Corp.) were added to each well. Then, 1 μL, of the COX2 Primer Probe MIX was added to each well, and contents of the well were mixed. After preparation of the PCR reaction mixture, the PCR plate was set to the ABI realtime PCR apparatus. The plate was processed in a thermal cycle at 95° C. for 10 minutes as the step 1, 95° C. for 15 seconds as the step 2, and 60° C. for 1 minute as the step 3, after heating at 50° C. for 2 minutes, and this thermal cycle was repeated 40 times. During the PCR reaction, fluorescence intensity was measured as a time course. FIG. 5 shows the result indicating the expression level (copy number) of the COX2 gene in the RNAs recovered from each sample. The expression level was calculated by analyzing the measured data of the fluorescence intensity. It was demonstrated that the higher the concentration of the sodium chloride, which was added to the simulated fecal sample, the higher the expression level of the COX2 gene in the RNA preparations. In particular, the expression level of the COX2 gene in the RNA recovered from the fecal sample 1E, which was left at room temperature for 24 hours, was higher than that in the RNA recovered from the fecal sample 1A. This result means that a highly pure human-originated nucleic acids containing extremely small amount of nucleic acids derived from bacteria was recovered in the method of recovery of the present invention. It is clear that accuracy of analysis of human-originated nucleic acids would be improved by using the nucleic acid preparation recovered in the method of recovery of the present invention.

### Example 2

One gram of the simulated fecal sample, which was prepared as in the Example 1, was aliquoted to each of seven 15 mL volume polypropylene tubes. By adding 10 mL of the saturated sodium chloride to each tube, fecal samples 2A to 2G were prepared. These samples were stored at 37° C. for various lengths of time, the simulated fecal samples being immersed in the saturated sodium chloride. Specifically, the lengths of time allowed to stand at 37° C. for the fecal sample 2A and 2B were 1 minute and 30 minutes, respectively. The fecal sample 2C was stored at 37° C. for 3 hours with mixing every 30 minutes. The fecal sample 2D was stored at 37° C. for 6 hours with mixing every 30 minutes. The fecal sample 2E was stored at 37° C. for 6 hours with mixing every 30 minutes and further allowed to stand at 37° C. for 6 hours (total 12 hours at 37° C.). The fecal sample 2F was stored at 37° C. for 6 hours with mixing every 30 minutes and further allowed to stand at 37° C. for 18 hours (total 24 hours at 37° C.). The fecal sample 2G was stored at 37° C. for 6 hours with mixing every 30 minutes and further allowed to stand at 37° C. for 66 hours (total 72 hours at 37° C.).

Each fecal sample was centrifuged immediately after a completion of the predetermined immersing time. Then, their supernatant was removed to obtain the solid contents. The solid contents were washed with sodium acetate buffer as in the Example 1, and then, RNAs were recovered from the washed solid contents.

The obtained RNA preparations were subjected to electrophoresis. After staining the gel, the band intensity of 16S rRNA, 23S rRNA, 18S rRNA, and 28S rRNA was measured. The band intensity was expressed as relative values relative to the intensity of 16S rRNA. FIG. 6 shows the result indicating the band intensity of each rRNA at the different points in time left at 37° C. in each fecal sample. From this result, it was demonstrated that only the bands representing rRNAs derived from a human were detected, while the bands representing rRNA derived from bacteria were disappeared, after immersing in the saturated sodium chloride for 12 hours. In addition, the band intensity after 1 minute at 37° C. was reduced by half in 6 hours in the rRNAs derived from a human, while it was reduced by half in 3 hours in those from bacteria. Base on the results explained above, it was confirmed that by immersing feces in the saturated sodium chloride, degradation of nucleic acids derived from bacteria was more enhanced than that from a human. In addition, it was demonstrated that a highly pure human-originated nucleic acids could be recovered by immersing feces in the treatment solution with a high salt concentration for a period long enough, even though the fecal samples used in this Example were the simulated fecal samples.

### Example 3

One gram of feces collected from a healthy individual was aliquoted to each of three 15 mL volume polypropylene tubes. Then, 10 mL of the saturated sodium chloride was added to each tube to prepare the fecal samples 3A to 3C. These samples were stored at room temperature (20° C.) for various lengths of time, the fecal samples being immersed in the saturated sodium chloride. Specifically, the fecal sample 3A was allowed to stand for 1 minute at 20° C. without mixing. The fecal sample 3B was allowed to stand for 18 hours at 20° C. after dispersing the feces by mixing sufficiently after addition of the saturated sodium chloride. The fecal sample 3C was allowed to stand for 36 hours at 20° C. after dispersing the feces by mixing sufficiently after addition of the saturated sodium chloride.

Each fecal sample was centrifuged immediately after completion of the predetermined immersing time. Then, their supernatant were removed to obtain the solid contents. PBS (phosphate buffered saline, pH7) was added to the solid contents as a washing solution. After suspending the solid contents sufficiently, the suspensions were centrifuged again. By removing the supernatants, washed solid contents were recovered. RNAs were recovered from the solid contents as described in the Example 1.

By using a part (5 μL) of each RNA solution, cDNAs were prepared with the ReverTra Ace qPCR RT Kit. Using the cDNAs as templates, nucleic acid amplification reactions were performed. The PCR solution included components detailed below. The PCR solution included the reverse transcribed cDNA, 12.5 μL of 2×TaqMan PCR master mix (manufactured by Parkin-Elmer Applied Biosystems), 900 nM of a forward primer for the human GAPDH gene (SEQ No. 1: 5′-GAAGGTGAAGGTCGGAGTC-3′), and 900 nM of a reverse primer for the human GDPHD gene (SEQ No. 2: 5′-GAAGATGGTGATGGGATTTC-3′), the final volume of the PCR solution adjusted to 25 μL. Using the PCR solutions prepared as explained above, the SYBR green PCR analysis was performed with the ABI Prism 7700 (manufactured by Parkin-Elmer Applied Biosystems). The PCR solutions were processed in a thermal cycle consisted of 95° C. for 30 seconds as the step 1, 55° C. for 30 seconds as the step 2, and 72° C. for 30 seconds as the step 3 after a denaturing cycle where the samples were heated at 95° C. for 10 minutes, and the cycle was repeated 45 times. The amplified products were quantified based on the fluorescent intensity data obtained from an amplification reaction using a dilution series of a standard plasmid DNA whose concentration has been known as the templates. When the RNA derived from the fecal sample 3A was used as the template, the amount of the amplified product was reduced less than 1/10 of the amplified product obtained by using the RNA derived from the fecal sample 3B. On the other hand, the difference of the amounts of the amplified products obtained from the fecal samples 3B and 3C was less than about 10%. Therefore, there was no drastic difference as seen in the comparison between the fecal samples 3A and 3B. As such, from the results explained above, it was demonstrated that the nucleic acids derived from human could be efficiently amplified from the nucleic acids prepared in the method of recovery of the present invention by immersing feces in the treatment solution for a certain length of time. This means that human-originated nucleic acids from feces were selectively recovered in the method of recovery of the present invention. In this Example, the amounts of nucleic acids derived from a human and bacteria were not compared side by side as in the Examples 1 and 2. Therefore, strictly speaking, the selective recovery effect of the present invention was not confirmed. However, it is reasonable to interpret that the human-originated nucleic acids contained in feces were preserved and recovered selectively as in the Examples 1 and 2, since the human-originated nucleic acid was amplified well.

### Example 4

One gram of feces collected from a healthy individual was aliquoted to each of eight 15 mL volume polypropylene tubes. Then, 6 mL of 26% saline solution (hereinafter referred as a high salt saline solution) was added to each tube, and the feces in the tubes was sufficiently suspended to prepare the fecal samples 4A to 4H. These samples were stored at room temperature (20° C.) for various lengths of time, the fecal samples being immersed in the high salt saline solution. Specifically, the fecal samples 4A and 4E were allowed to stand for 1 hour. The fecal samples 4B and 4F were allowed to stand for 24 hours. The fecal samples 4C and 4G were allowed to stand for 72 hours. The fecal samples 4D and 4 H were allowed to stand for 168 hours.

Each fecal sample was centrifuged immediately after a completion of the predetermined immersing time. Then, their supernatant was removed to obtain the solid contents. RNAs were recovered from the solid contents as described in the Example 1.

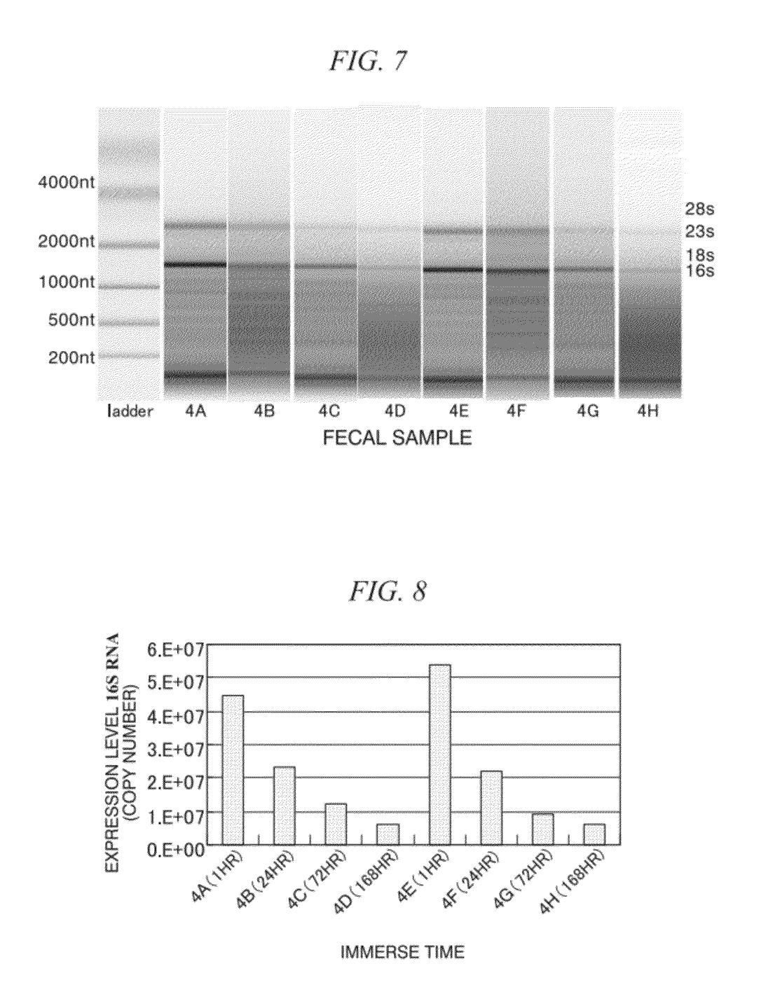

The obtained RNA solutions were subjected to electrophoresis. After the electrophoresis, the gel was stained and bands representing RNA populations were visualized. The obtained image of the stained gel is shown in FIG. 7. In FIG. 7, the lane labeled as “ladder” is the lane in which a size maker was run. In TABLE 2, relative band intensity of bands representing 16S rRNA and 23S rRNA in the stained image is shown.

From the result shown in Table 2, it was confirmed that the longer the immersion time, the more 16S and 23S rRNAs were degraded. In this Example, the original amount of 18S and 28S rRNAs were too low to be detected, even after the longer immersion time.

TABLE 2

Band Intensity
of rRNA
(Relative value)

| Fecal samples | 16S | 23S |
|---|---|---|
| 4A | 633.5 | 186.2 |
| 4B | 627.8 | 172.2 |
| 4C | 480.9 | 47.5 |
| 4D | 198.4 | 22.6 |
| 4E | 683.4 | 179.5 |
| 4F | 575.6 | 297.4 |
| 4G | 449.1 | 62.4 |
| 4H | 174.2 | 16.4 |

Using the obtained RNAs from the fecal samples as a template, cDNAs were prepared by performing reverse transcription reactions. The amount of RNAs in each reverse transcription was adjusted to 1 μg. Using the obtained cDNAs, realtime PCR reactions were performed with a forward primer for the bacterial 16S rRNA (SEQ No. 3: 5′-AGGAGGTGATCCAACCGCA-3′) and a reverse primer for the bacterial 16S rRNA (SEQ No. 4: 5′-AACTGGAGGAAGGTGGGGAT-3′) to measure the expression level of 16S rRNA of Escherichia coli. FIG. 8 shows the result indicating the calculated expression level (copy number) of 16S rRNA gene in the RNA preparations recovered from each fecal samples. The number in the parentheses after the names of the fecal samples indicates the immersion time that each fecal sample was immersed in the high salt saline solution. Based on the result, it was confirmed that the longer the immersion time, the less expression level of 16S rRNA gene.

### Example 5

Nucleic acids were recovered from fecal samples prepared with a fecal sample container equivalent to one shown in FIG. 3.

First, with the bar for collecting feces 3, about 0.5 g of feces was collected in the cup 3a. Then the lid was closed to insert the feces into the container, resulting in the fecal sample 5A. The fecal sample 5B is a reference sample and prepared as described below. About 0.5 g of feces was collected in a 15 mL volume polypropylene tube containing 5 mL of the saturated sodium chloride. The volume ratio between the feces and the treatment solution with a high salt concentration was 1:10, as in the fecal sample 5A. After storing the fecal sample 5A and the reference sample 5B at 30° C. for 18 hours, RNAs were recovered from the samples. From the RNA preparations, cDNAs were prepared as in the Example 3. Then, by using the prepared cDNA, quantitative PCR of the human GAPDH gene was performed. When comparison was made between the amplification products from the RNAs of the fecal sample 5A and the reference sample 5B, amplification efficiency of the amplified product from the sample 5B was 10% less than that from the fecal sample 5A. It was assumed that a more highly pure human-originated nucleic acid was recovered in the fecal sample 5A by using the fecal sample container, which is equivalent to one shown in FIG. 3, since the feces can be quickly immersed in the treatment solution with the container. In addition, it is possible to reduce the labor costs spent to the operators on the examining process by using the fecal sampling container as shown in FIG. 3. The reason for that is the collector of the feces can perform the preparation and immersing of the fecal sample easily, immediately after the collection.

The fecal samples container is just one embodiment of the present Example. Therefore, the shape of the container may not be the shape described above as long as the feces can be collected quickly, and a certain amount of the feces can be immersed in the treatment solution with a high salt concentration quickly.

### Example 6

One gram of feces collected from a healthy individual was aliquoted to each of three 15 mL volume polypropylene tubes. Six milli litter of the saturated sodium chloride was added to all the 3 tubes. The feces in the tubes was sufficiently suspended to prepare the fecal samples 6A to 6C. All the fecal samples were allowed to stand for 24 hours immersing the feces in the treatment solution at various temperatures. The temperature for the fecal samples 6A, 6B, and 6C were 4° C., 16° C., and 30° C., respectively.

Each fecal sample was centrifuged immediately after a completion of the predetermined immersing time. Then, their supernatant were removed to obtain the solid contents. From the solid contents, RNAs were recovered as described in the Example 2. From the recovered RNAs, cDNAs were prepared, and the quantitative PCR for the human GAPDH gene was performed as described in the Example 3. Based on the quantitative PCR, the expression level (copy number) of the human GAPDH gene obtained from each fecal sample was calculated. The copy number calculated from the analysis using the RNA from the fecal sample 6A as the template was 6,200 copies. Ones from the fecal sample 6B and 6C were 94,000 copies and 153,000 copies, respectively. Thus, in terms of the immersing temperature, the higher temperature is preferable in the temperature range of 4° C. to 30° C.

### Example 7

Nucleic acids were recovered after immersing feces in various concentrations of ammonium sulfate.

First, 0% (water), 10%, 20%, 30%, 40%, and the saturated solution of ammonium sulfate were prepared. Then, each of the ammonium solution (10 mL) was poured into six 15 mL volume polypropylene tubes separately. Depending on temperature, precipitations of ammonium sulfate were seen in the saturated solution.

One gram of the simulated feces was aliquoted to each tube as described in the Example 1. Then, the feces was suspended sufficiently to prepare the fecal samples. The fecal samples were centrifuged after incubation at 20° C. for 24 hours. Then, the solid contents were recovered by removing the supernatants. RNAs were prepared from the solid contents as described in the Example 1. The obtained RNA preparations were subjected to electrophoresis. The bands representing RNA populations were visualized by staining the gel.

From the image of the stained gel, the RNA bands representing 16S and 23S rRNA were clearly visible in the fecal samples including 20% or less of ammonium sulfate (including the solution without ammonium sulfate (0% ammonium solution)). However, all the RNA bands representing 18S and 28S rRNAs were smeared.

On the other hand, in the RNAs recovered with the fecal sample using 30% ammonium sulfate, the RNA bands representing 18S and 28S rRNAs were visible with a weak intensity. Also, degradation of 16S and 23S rRNAs was observed. In the RNAs recovered with the fecal sample using 40% or more of ammonium sulfate, the RNA bands representing 18S and 28S rRNAs were visible more clearly. Also, more degradation of 16S and 23S rRNAs was observed. Based on the results explained above, it was demonstrated that the lowest concentration of ammonium sulfate to obtain the selective recovery effect of the present invention was 30 to 40%, when ammonium sulfate was used as the active component of the treatment solution with a high salt concentration.

The lowest concentration of the salt, which is the active component of the treatment solution, can be changed depending on conditions, such as the type of the salt, the size of the feces piece, the immersion temperature, and the immersion time. In a harsher temperature condition, even higher concentration of ammonium sulfate would be needed.

### Example 8

In this Example, effects of pH, temperature, and salt concentration of the treatment solution to the selective recovery effect of the present invention were investigated.

First, 1 g of the simulated fecal sample, which was prepared as described in the Example 1, was aliquoted to each of eight 15 mL volume polypropylene tubes. The four ammonium sulfate solutions, which had varying concentrations of ammonium sulfate and pH, were prepared. The first solution included 30% ammonium sulfate and its pH was 7.0. The second solution included 30% ammonium sulfate and its pH was 5.0. The third solution included 40% ammonium sulfate and its pH was 7.0. The fourth solution included 40% ammonium sulfate and its pH was 5.0. Each of these ammonium sulfate solutions were poured into two of the above-mentioned polypropylene tubes holding the simulated fecal samples. Then, the feces was suspended sufficiently to prepare the fecal samples. The 8 tubes were divided in half (four tubes per one set), each set including the first, second, third and fourth solutions. One set was allowed to stand at 37° C. and another at 25° C. (room temperature) for 24 hours. After immersing for the predetermined period of time, the fecal samples were centrifuged. Then, the solid contents were recovered by removing their supernatants. RNAs were recovered from the solid contents as described in the Example 2. The obtained RNA preparations were subjected to electrophoresis. The bands representing nucleic acid populations were visualized by staining the gel.

Based on the result obtained by the electrophoresis analysis, the following was demonstrated. In a case where the fecal sample was treated with the first solution (30% ammonium sulfate at pH 7.0) at 25° C., the selective recovery effect of the present invention was obtained. However, when the immersion was performed at 37° C. with the same ammonium solution (the first solution), the ratio of 18S and 28S rRNA was not increased in the RNA preparations. When the ammonium concentration was 40%, the treatment solution having pH 5.0 gave improved selective recovery effect of the present invention compared to the treatment solution having pH 7.0. Therefore, at a higher concentration of ammonium sulfate (40%), human rRNAs were recovered more preferentially at a lower pH (5.0). Changing only one parameter (increasing the ammonium sulfate concentration from 30 to 40%, or lowering the pH from 7.0 to 5.0) did not notably improve the stability of human-originated RNAs and the selective recovery effect.

While preferred embodiments of the invention have been described and illustrated above, it should be understood that these are examples of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

Nucleic acids derived from mammalian cells can be selectively recovered from feces, which includes mammalian and bacterial cells, in the method of recovery of the present invention. Therefore, the method of recovery of the present invention can be applicable the clinical examination or the like using fecal samples, such as a periodic medical examination.

What is claimed is:

1. A method of recovering a nucleic acid derived from a mammalian cell taken from feces comprising the steps of:
(A) immersing feces, in which feces is immersed in a treatment solution with a high salt concentration for a predetermined period of time to prepare a fecal sample;
(B) recovering a solid component, in which a solid component is recovered from the fecal sample after the step (A); and
(C) recovering a nucleic acid from the solid component recovered in the step (B).

2. The method of recovering a nucleic acid derived from a mammalian cell taken from feces according to claim 1,
wherein, the treatment solution with a high salt concentration is a solution containing one or more salts selected from the group consisting of sodium chloride and ammonium sulfate.

3. The method of recovering a nucleic acid derived from a mammalian cell taken from feces according to claim 1,
wherein, the treatment solution with a high salt concentration contains sodium chloride, the concentration of which is 13% (wt/wt) or higher and the saturating concentration of sodium chloride or lower.

4. The method of recovering a nucleic acid derived from a mammalian cell taken from feces according to claim 1,
wherein, the treatment solution with a high salt concentration contains sodium chloride, the concentration of which is 20% (wt/wt) or higher and the saturating concentration of sodium chloride or lower.

5. The method of recovering a nucleic acid derived from a mammalian cell taken from feces according to claim 1,
wherein, the treatment solution with a high salt concentration contains ammonium sulfate, the concentration of which is 30% (wt/wt) or higher and the saturating concentration of ammonium sulfate or lower.

6. The method of recovering a nucleic acid derived from a mammalian cell taken from feces according to claim 1,
wherein, a salt concentration of the treatment solution with a high salt concentration is 80% of the saturating concentration of the salt or higher and the saturating concentration of the salt concentration or lower.

7. The method of recovering a nucleic acid derived from a mammalian cell taken from feces according to claim 1,
wherein, a salt concentration of the treatment solution is the saturating concentration of the salt.

8. The method of recovering a nucleic acid derived from a mammalian cell taken from feces according to claim 1,
wherein, the treatment solution with a high salt concentration contains two or more kinds of salts.

9. The method of recovering a nucleic acid derived from a mammalian cell taken from feces according to claim 1,
wherein, the treatment solution with a high salt concentration contains a chelating agent that complexes with a divalent cation.

10. The method of recovering a nucleic acid derived from a mammalian cell taken from feces according to claim 1,
wherein, the pH value of the treatment solution with a high salt concentration ranges from 4 to 8.

11. The method of recovering a nucleic acid derived from a mammalian cell taken from feces according to claim 1,
wherein, the treatment solution with a high salt concentration has a buffer action.

12. The method of recovering a nucleic acid derived from a mammalian cell taken from feces according to claim 1, further comprising the step of:
(D) washing the solid component recovered in the step (B) to reduce the salt concentration of the solid component, after the step (B) and before the step (C).

13. The method of recovering a nucleic acid derived from a mammalian cell taken from feces according to claim 12,
wherein, the salt concentration of the solid component after the step (D) is less than 100 mM.

14. The method of recovering a nucleic acid derived from a mammalian cell taken from feces according to claim 12,
wherein, the salt concentration of the solid component after the step (D) is less than 30 mM.

15. The method of recovering a nucleic acid derived from a mammalian cell taken from feces according to claim 12,
wherein, the step (D) comprises the steps of:
(d) dispersing the solid component in a washing solution; and
(e) recovering a washed solid component by centrifuging the washing solution dispersing the solid component and removing a supernatant after the centrifugation to recover the washed solid component, and
the washing solution is a solution selected from the group consisting of a buffer solution with a low ion concentration, a water-soluble organic solvent, water, and a mixture thereof.

16. The method of recovering a nucleic acid derived from a mammalian cell taken from feces according to claim 1,
wherein, the fecal sample is agitated at least once in the step (A).

17. The method of recovering a nucleic acid derived from a mammalian cell taken from feces according to claim 1,
wherein, the feces is immersed in the treatment solution with a high salt concentration for 12 hours or longer.

18. The method of recovering a nucleic acid derived from a mammalian cell taken from feces according to claim 17,
wherein, the feces is immersed in the treatment solution with a high salt concentration for 24 hours or longer.

19. The method of recovering a nucleic acid derived from a mammalian cell taken from feces according to claim 18,
wherein, the feces is immersed in the treatment solution with a high salt concentration for 72 hours or longer.

20. The method of recovering a nucleic acid derived from a mammalian cell taken from feces according to claim 1,
wherein, the immersing of the faces in the treatment solution is performed at 4° C. or higher.

21. The method of recovering a nucleic acid derived from a mammalian cell taken from feces according to claim 20,
wherein, the immersing of the faces in the treatment solution is performed at 10° C. or higher.

22. The method of recovering a nucleic acid derived from a mammalian cell taken from feces according to claim 21,
wherein, the immersing of the faces in the treatment solution is performed at 16° C. or higher.

23. The method of recovering a nucleic acid derived from a mammalian cell taken from feces according to claim 1,
wherein, 1 volume of the feces is mixed with 1 volume or more of the treatment solution with a high salt concentration in the step (A).

24. The method of recovering a nucleic acid derived from a mammalian cell taken from feces according to claim 1,
wherein, the treatment solution with a high salt concentration includes a surface-activating agent.

25. The method of recovering a nucleic acid derived from a mammalian cell taken from feces according to claim 1,
wherein, the treatment solution with a high salt concentration includes a coloring agent.

26. The method of recovering a nucleic acid derived from a mammalian cell taken from feces according to claim 1,
wherein, the nucleic acid derived from a mammalian cell and the nucleic acid derived from an enterobacterial cell are recovered concurrently from the solid component.

27. The method of recovering a nucleic acid derived from a mammalian cell taken from feces according to claim 1,
wherein, the step of recovering the nucleic acid from the solid component in the step (C) further comprises the steps of;
(a) eluting, in which the nucleic acid is eluted from the enterobacterial cell and the mammalian cell in the solid component by denaturing a protein in the solid component; and
(b) recovering eluted nucleic acid, in which the nucleic acid eluted in the step (a) is recovered.

28. The method of recovering a nucleic acid derived from a mammalian cell taken from feces according to claim 27 further comprising the step of;
(c) removing protein, in which the protein denatured in the step (a) is removed after the step (a) and before the step (b).

29. The method of recovering a nucleic acid derived from a mammalian cell taken from feces according to claim 27,
wherein, the denaturing of the protein in the step (a) is performed by using one or more selected from the group consisting a chaotropic salt, an organic solvent, and a surface-activating agent.

30. The method of recovering a nucleic acid derived from a mammalian cell taken from feces according to claim 29,
wherein, the chaotropic salt is a guanidine salt.

31. The method of recovering a nucleic acid derived from a mammalian cell taken from feces according to claim 28,
wherein, the removing of protein in the step (c) is performed by using chloroform.

32. The method of recovering a nucleic acid derived from a mammalian cell taken from feces according to claim 27,
wherein, the step (b) for recovering the eluted nucleic acid comprises the steps of:
(b1) adsorbing nucleic acid, in which the nucleic acid eluted in the step (a) is absorbed to an inorganic support body; and
(b2) eluting absorbed nucleic acid, in which the nucleic acid absorbed in the step (b1) is eluted from the inorganic support body.

33. A nucleic acid recovered from feces by using the method of recovering a nucleic acid derived from a mammalian cell according to claim 1.

34. A method of analyzing a nucleic acid derived from a mammalian cell using the nucleic acid recovered by the method of recovering a nucleic acid derived from a mammalian cell according to claim 1.

35. The method of analyzing a nucleic acid derived from a mammalian cell according to claim 34 wherein the mammalian cell is a gastrointestinal cell.

36. The method of analyzing a nucleic acid derived from a mammalian cell according to claim 34 wherein the mammalian cell is a colonic exfoliated cell.

37. The method of analyzing a nucleic acid derived from a mammalian cell according to claim 34 wherein the nucleic acid derived from a mammalian cell is a marker of neoplastic conversion.

38. The method of analyzing a nucleic acid derived from a mammalian cell according to claim 34 wherein the nucleic acid derived from a mammalian cell is a marker of inflammatory gastrointestinal disease.

39. The method of analyzing a nucleic acid derived from a mammalian cell according to claim 34 wherein the nucleic acid derived from a mammalian cell is a nucleic acid derived from the COX2 (cyclooxygenase-2) gene.

40. The method of analyzing a nucleic acid derived from a mammalian cell according to claim 34 wherein one or more of analyses selected from the group consisting of the expression analysis of mRNA, the mutational analysis of the K-ras gene, and, the DNA methylation analysis are performed.

41. The method of analyzing a nucleic acid derived from a mammalian cell according to claim 40,
wherein a portion of a total RNA recovered from the feces is reverse transcribed to convert the total RNA to cDNA,
a nucleic acid amplification reaction is performed using the cDNA as a template for, and
a product of the nucleic acid amplification reaction is analyzed in the expression analysis of mRNA.

42. A kit for feces sampling comprising:
a treatment solution having high salt concentration; and
a feces sampling container holding the treatment solution having high salt concentration.

43. The kit for feces sampling according to claim 42 further comprising:
a washing solution.

44. A fecal sample processing apparatus comprising:
a solution removing device that removes the treatment solution having high salt concentration from the feces sample that stored for a predetermined period by immersing the sampled feces in the treatment solution having high salt concentration.


SEQUENCE LISTING (4 sequences)

SEQ ID NO: 1 - DNA, 19 bp, Artificial Sequence
  Description of Artificial Sequence Forward primer for h-GAPDH.
gaaggtgaaggtcggagtc

SEQ ID NO: 2 - DNA, 20 bp, Artificial Sequence
  Description of Artificial Sequence Reverse primer for h-GAPDH.
gaagatggtgatgggatttc

SEQ ID NO: 3 - DNA, 19 bp, Artificial Sequence
  Description of Artificial Sequence Forward primer for 16S rRNA.
aggaggtgatccaaccgca

SEQ ID NO: 4 - DNA, 20 bp, Artificial Sequence
  Description of Artificial Sequence Reverse primer for 16S rRNA.
aactggaggaaggtggggat